(19) United States
(12) Patent Application Publication
(10) Pub. No.: US 2014/0342443 A1
(43) Pub. Date: Nov. 20, 2014

(54) PHOTO-BIOREACTOR (Algae Greenhouse-Dry Cooling Tower)

(71) Applicant: Donovan Dale Bohn, Forest Lake, MN (US)

(72) Inventor: Donovan Dale Bohn, Forest Lake, MN (US)

(21) Appl. No.: 13/897,286

(22) Filed: May 17, 2013

(51) Int. Cl. C12M 1/00

(52) U.S. Cl. CPC C12M 21/02; C12M 39/00; C12M 31/02
USPC 435/292.1

(57) ABSTRACT

This closed system Photo-Bioreactor (PBR) system includes all of the necessary equipment to naturally collect solar energy and through photosynthetic process produces useable algal biomass. This PBR utilizes waste heat and waste CO2 exhaust from power plants or other industrial sources plus waste or other nutrients from domestic, agriculture or other systems. Because this system operates in a closed loop, environmental contamination of the algae is avoided. The PBR inherently performs as a dry cooling tower saving precious water otherwise evaporated from wet cooling towers. The PBR uses transparent tubing with external opaque stripes to optimize light absorption for enhanced algae growth. The PBR enclosure uses pneumatically operated bladder curtains to allow cold weather operation. Low abrasion pumps are used to circulate the reagent solution in the system. Algae is harvested via filtration or other means when concentrations are saturated.

### FIELD OF INVENTION

The subject of this application relates generally to production of algae and more specifically to a system and method for production of algae.

### DETAILED DESCRIPTION

The following detailed description is exemplary in nature and is not intended to limit the scope, applicability, or configuration of the invention in any way. Rather, the following description provides practical illustrations for implementing exemplary embodiments of the invention.

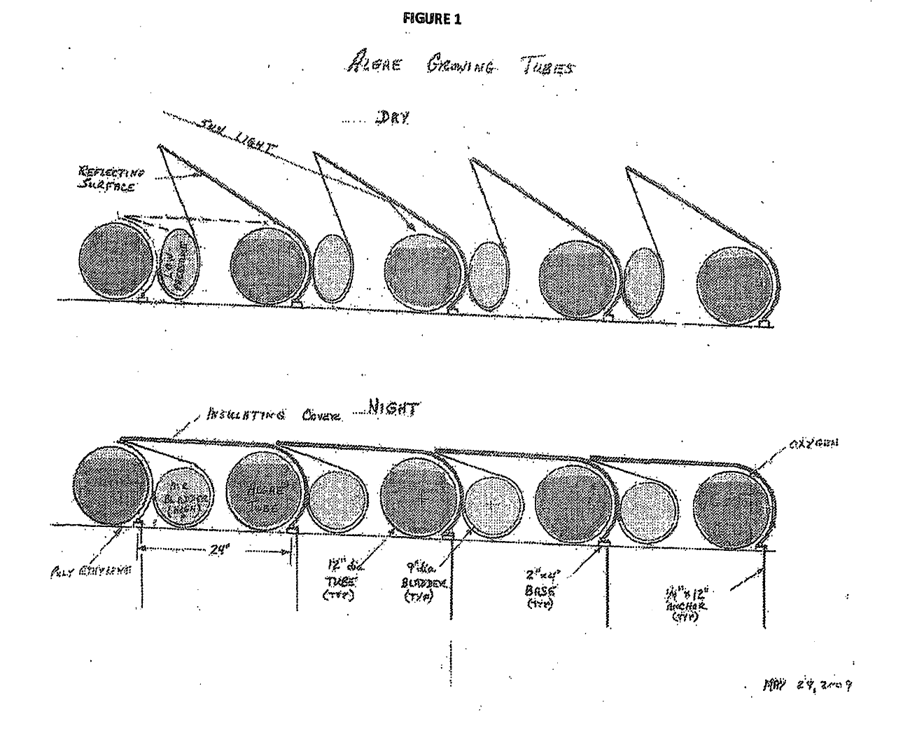

Referring to FIG. 1 an exemplary embodiment of the invention is shown in cross-sectional view. The photo-bioreactor (PRB) is shown. As shown, a plurality of translucent or transparent tubes is provided. The tubes are made of glass or a plastic such as Plexiglas. The tubes contain algae, water, and nutrients that may be circulated with pumps. The tubes may be positioned in an array such as layers of ten tubes spaced at six inch intervals horizontally and fourteen layers of tubes spaced at six inch intervals vertically. Multiple arrays of tubes may be positioned side-by-side spaced thirty inches apart. The space between arrays may provide a walkway down the length of the tubes.

As sunlight travels through algae containing liquids, the light is absorbed by the algae. The depth of algae culture through which sunlight will penetrate depends on the concentration of the algae in the solution. The diameter of the tubes is chosen to maximize the algae production per unit of capital invested. For some algae cultures, sunlight only penetrates about one inch past the surface, In some algae cultures, the optimum tube size is about two inch inside diameter. In larger diameter tubes, all of the incident sunlight is absorbed by the algae before the light can penetrate to the center of the tube. Smaller diameter tubes have higher capital cost per unit of algae production and higher pumping energy cost.

The arrays of tubes are enclosed in a structure with roof panels and apertures. The roof panels may be sloped downward towards the north at 45 degrees. The roof panels may be made of concrete or other structural material. Between adjacent roof panels, apertures are positioned to allow sun light to enter. The apertures are made of a translucent or transparent material such as glass a plastic like Plexiglas,

During algae production, water, algae, nutrients, and a carbon dioxide carrier such as sodium bicarbonate, are introduced into the tubes. These may be introduced from ancillary sources, e.g., containers or dispensers on site. For example, in some applications, carbon dioxide may be collected from a carbon dioxide scrubber and directed to the PBR. Nutrients may be purchased from and delivered by vendors or may be received as a by-product from other process streams or systems.

The rood panels are designed to provide shade in the summer to avoid overheating the enclosures during hot weather. The apertures are fitted with insulating curtains which are mechanically, electrically, or pneumatically operated. Insulating curtains are positioned against the inside of the apertures during hot or cold weather to maintain the temperature within the enclosure within the range in which algae grows. The curtains are moved away from the apertures during daylight hours to admit sunlight. At night during cold weather, the curtains are positioned against the apertures to reduce heat loss. On hot days, the curtains may be positioned against the apertures during portions of the day to reduce heat gain within the enclosure.

In the embodiment illustrated in FIG. 1, the insulating curtain is moved against and away from the apertures with pressurized air (pneumatic) bladders. Here low pressure opens the curtains, moving them away from the aperture and high pressure in the bladders closes the curtains, moving the curtains against the apertures. Other mechanisms may be used to control the positions of the insulating curtains.

Algae grow best with sunlight that is not as intense as continuous, direct sunlight. An example is Spirulina which grows best in light that has an average intensity of between 10% and 20% of full sunlight. Algae is known to grow faster when subjected to light/dark cycles that occur at a frequency of greater than one cycle per second. Some of the tubes in the PBR may be coated with opaque rings at intervals to provide an average light intensity which is particularly suited to the algae strain chosen for the PBR. An example of an exemplary embodiment is the top three layers of tubes are painted with a series of bright white rings one inch wide and ¼ inch apart. Sunlight enters the tubes along 20% of the length of the tubes, At velocities of more than twenty feet per minute, the algae in the solution passing through the tubes experiences light/dark cycles with a frequency of about four cycles per second. In the layers of tubes below the first three layers, light intensity is greater than it would otherwise be due to the light that has been reflected from the bright white painted rings above. The fourth, fifth, and sixth layers of tubes from the top of the array may have painted rings that are ¾ inch wide spaced inch apart. Sunlight that is primarily reflected light which is less intense that direct sunlight, enters the tubes along 60% of the length of the tubes. Algae experiences light/dark cycles with a frequency of about four cycles per second. The optimum width and spacing of painted rings is chosen taking into account the latitude of the site, the algae strain in the culture, and the average weather conditions.

Galleries may be disposed on opposing ends of the tubes and positioned at intervals along the length of the tubes. The galleries contain tanks, at least one sending tank and at least one receiving tank. Some of the tanks are equipped with pumps to facilitate circulation of algae and water through the tubes. Any pump suitable for moving algae containing liquids may be used. An example of a suitable pump is an auger style pump.

The galleries may include a device to de-oxygenate the algae containing liquid, As algae grow, it absorbs carbon dioxide and releases oxygen. As the concentration of oxygen increases, it will become toxic to the algae. At intervals along the length of the tubes, the oxygen must be vented. An oxygen venting arrangement may be built into the tanks. One example of such an arrangement is a tank with a receiving side and a sending side. Tubes bringing algae containing liquids terminate at pipe nipples protruding from the receiving side. The algae containing liquid flows from the tubes into pipe nipples protruding from the receiving side of the tank, up towards the top of the tank, horizontally across a shallow flow path, down into the sending side of the tank, and out pipe nipples protruding from the sending side of the tank. Oxygen is vented from the liquid as it flows across the shallow flow path.

Venting may be improved by bubbling air or oxygen up through the liquid as it flows across the shallow flow path. Pumping energy is minimized by bubbling air under a shallow stream of water. The oxygen vented from the top of the tanks may be vented to atmosphere, or it may be captured for a variety of uses. One such use would be to gasify coal in a power plant.

Variations on the PBR are described below, including construction techniques and ancillary systems and equipment that may be part of a larger integrated facility system and process.

In various embodiments of the invention, the PBR may further include, be configured with, or connected to solar concentrators, seasonal geothermal storage components and cycles, galleries; head tanks, and special pumps, algae and water circulation patterns, and water conservation systems or features, e.g. the PBR may operate as a dry cooling tower. It should be understood that the dimensions and specifications of various systems, components; and parameters are illustrative and they may be varied as design choice or process or economic efficiencies require.
1. The land surface is prepared and areas of predetermined dimensions, e.g. 10,000 ft×100 ft, are rolled smooth with no sharp objects protruding from the surface. The surface for some embodiments may be leveled to +/−6 inches in any 600 ft length.
2. In one example, a suitable flooring material may be polymeric sheets with a white or reflective surface in rows about 8 feet wide and 600 feet long, may be unrolled between concrete foundations for the tanks.
3. Foundation pieces (e.g. 2 in×6 in standard wood or steel studs) may be laid in a north-south orientation spaced ten feet apart and anchored to the earth with rods.
4. Tanks are positioned on concrete foundations at 600 foot intervals.
5. Precast concrete vertical supports are fastened to the foundation pieces. In one example, these supports are five feet wide and eight feet tall with three inch diameter holes to match the 10 wide×14 high array of tubes on six inch centers. The supports may include reinforcing wire or rods or other reinforcing material. The supports include a flanged bottom surface that rests on the foundation pieces and flanged top surfaces that support the roof sections. The roof support surfaces are slanted down at forty five degrees to the north. The support panels are braced to remain vertical.
The precast support panels are rapidly cured in high humidity and elevated temperature curing rooms for rapid production. The panels may include reinforcing rods, wires or other reinforcement.
6. Precast concrete roof panels are fastened to the vertical supports. In one example, these roof panels are twenty feet long and four feet wide with grooves to accept aperture plates. Insulation may be attached to the lower surface of the roof panels. The insulation may be in strips on part or the entire lower surface.
7. Apertures of translucent or transparent panels of glass or plastic are positioned between adjacent roof panels. The aperture panels may fit into grooves cast into the roof panels. In one example the apertures are double pane glass panels two feet wide and six feet long. The glass panels are positioned sloping upwards at forty five degrees from the roof panel on the south to the roof panel on the north.
8. Insulating curtains are prepared using bladders and insulating panels. Bladders made from polyethylene sheets or other flexible material, are attached to the north portion of the lower surface of each roof panel. The south edge of the bladder is attached to the roof panel along a line slightly to the north of the top edge of the apertures. A rigid insulating panel is attached to the bladder, so that when the bladder is inflated, this panel rotates toward the aperture.
9. After a tank and the associated vertical supports, roof panels, apertures, and insulating curtains are assembled to the west of the tank, before the next tank is placed, glass tubes are inserted. These tubes about two inch diameter and ten feet long are positioned at the end of PBR to the west of from the tank. One end of the tubes has been previously dipped in a liquid rubber or plastic coating material. This coating is allowed to harden. Ten tubes are placed side-by-side on rollers and pushed to the east a distance equal to the length of the tubes aligned with the lowest layer of holes in the closest vertical support panel. Another set of ten tubes is placed is position where the first tubes were initially placed. The rubber coating previously applied to the tube ends forms a cushion between the glass ends of the tubes. The second set of tubes is pushed toward the vertical support panel. At the interface between the two sets of tubes, a three inch wide strip of fiber reinforced heat shrink plastic is wrapped around each pair of tubes to be coupled together. The heat shrink plastic is welded to form a tube and cut off from the strip, The tubes of heat shrink plastic are heated and shrunk to form a water tight seal between the two tubes. The process is repeated until the first set of tubes reaches the pipe nipples protruding from the tank. The leading ends of the first tubes are connected to the pipe nipples on the tank with heat shrink plastic. The process is repeated with each succeeding layer of tubes until the entire height of the array is in place. The next tank is placed in position to the east of the PBR, so that the protruding pipe nipples are thirty inches from the west end of the tubes in the PBR array. Short lengths of glass tube are positioned between the tubes in the array and the pipe nipples on the west side of the tank and connected the glass tubes and pipe nipples with heat shrink plastic. This process is repeated until the entire length of the PBR is assembled.
Glass tubes are delivered to the tube insertion equipment in cassettes. Each cassette may hold a column of thirty tubes horizontally, one above the other, on a conveyor system. The conveyor rotates intermittently to position tubes onto the insertion equipment. Ten such conveyors may be assembled side-by-side into one cassette, so that ten tubes are simultaneously placed in position to be inserted. When the train of tubes is pushed forward, rollers guide the glass tubes into each successive supporting panel.
Roof panels may be placed over the tanks to create a continuous roof over the entire length of the PBR.
10. Pumps are installed in the tanks as required. For example the pumps may be installed at 1200 foot intervals (on every other tank in a row). The pumps may be auger style for example with a vertical shaft and a variable speed gear motor drive. The auger may be enclosed in a twelve inch vertical pipe
11. The bladders are connected to fans with pipe or ducts. The fans are arranged so that air can be blown into the bladders to inflate them and out of the bladders to deflate them.
12. Drain lines similar to drains on a house eve gutter are connected to the roof panels at appropriate intervals. These lines may be connected to a rain water collection system and used to capture the rain water.
13. When the PBR is in operation, a continuous cleaning system may be used to keep algae from coating the inner surface of the tubes. Balls slightly smaller than the interior diameter of the tubes and of nearly neutral buoyancy are circulated with the algae containing fluid. The balls continuously rub the tube walls and remove any algae or film that might build up.
14. Filters or other equipment are installed to remove harvest the algae from the PBR. The amount of algae removed each day is approximately the same as the amount that has grown that day. In a preferred embodiment, the alga is a strain of Spirulina. Since the Spirulina algal cells are relatively long and easily removed with a filter belt, a horizontal belt filter is used to harvest the algae. Fresh water is used to wash the algae after most of the liquid has been removed by the filter. This adds make-up water to replace the water used by the algae and reduces the amount of salts and nutrients that are removed with the algae.

WATER and ALGAE CIRCULATION PATTERN

The temperature of the PBR is kept high enough for the algae to grow in the winter by using waste heat, tor example waste heat from a power plant that is normally dispersed into the atmosphere with a cooling tower. The PBR will conserve water that otherwise is used in an evaporative cooling tower to remove the heat. The PBR then serves as a dry cooling tower.

The concentration of sodium bi-carbonate will typically vary between locations in the PBR. The maximum sodium bi-carbonate concentration will occur at the scrubber outlet. As this solution is introduced into the PBR it will be diluted to the “operating” level. The concentration will highest the morning. During the day, the concentration will decrease as the algae consume carbon dioxide at a rate higher than it is being delivered from the scrubbers. During the night, the concentration will rise as more carbon dioxide is delivered while the algae are dormant. As algae consume carbon dioxide, sodium bi-carbonate is converted to sodium carbonate. In the carbon dioxide scrubber, the sodium carbonate is converted back into sodium bi-carbonate.

WATER CONSERVATION FEATURES Dry Cooling Tower

The sensible heat demand associated with a PER that is integrated with a power plant according to embodiments of the invention may require 100% of the heat rejected from the plant's turbine generator condenser/circulating water system plus the maximum that is reasonably available in the plant's flue gas during much of the year. The PER may not be able to absorb all of this heat during hot summer weather in which case the excess waste heat will be rejected through the cooling tower system.

This sensible heat rejection inherently available in the invention creates three primary Water Conservation Features.
a. The plant cooling tower operations are either significantly reduced or totally shutdown to supply the majority of the heat load. This results in a very large scale Combined Heat and Power (CHP) application. Each MW-yr generated where the associated turbine generator heat rejection is diverted to algae production, conserves about 8.5 acre feet, or 2.5 million gallons of water per year.
b. Because the CO2 scrubber cools the flue gas, more than 50% of the entrained water vapor condenses. The water vapor originates from fuel hydrogen and from moisture in the combustion air and fuel. For a typical low sulfur western coal, each MW-yr saves about an additional 3 acre feet or one million gallons per year of water.

In the foregoing detailed description, the invention has been described with reference to specific embodiments. However, various modifications and changes can be made without departing from the scope of the invention as set forth in the appended claims.

We claim:

1. A cover over the growing tubes that shades the tubes from excessive heat in summer.

2. A cover made of roof panels that slope upward toward the south with sufficient over-hang to provide shade during hot summer days and admit full sunlight during winter days.

3. South facing apertures that admit sunlight and enclose the PBR to facilitate control of the interior temperature.

4. Means of opening solar apertures using pneumatically operated bladders to move Insulating curtains that insulate the aperture during cold or hot weather. The insulating curtains control light and heat flux through long apertures (possibly 700 feet long) by pumping air into and releasing air from the bladders.

5. Transparent tubes in which algae grows as it flows from end to end. Relatively short segments of tubes are joined into long lengths using heat shrink plastic. The heat shrink plastic may be fiber reinforced. The heat shrink plastic may be made of several plies.

6. Transparent tubes in which algae grows that have opaque rings painted at intervals such that the algae flowing through the tubes experiences light/dark cycles of greater than one cycle per second. The pattern of ring width and space between adjacent rings is chosen to expose the algae to the optimum average intensity of light for rapid algae growth. The pattern of rings varies from top to bottom in the array of tubes.

7. The array of tubes is supported by vertical supports such as concrete panels with holes for each rube.

8. Tanks at intervals along the length of the tubes with means to vent or collect oxygen.

9. Pumps at intervals along the length of the tubes that are designed to move algae containing fluid with minimal damage to the algae, such as auger style pumps.

10. The heat demand required to sustain optimum algae/microbiology growing in both cool and warm latitudes is an effective substitute for the cooling towers currently utilized by host facilities that implement this integrated CO2-algae growing technology. This alternative to cooling tower heat rejection represents a tremendous water conservation benefit (as noted in the description) for these host facilities.

11. The system utilizes a comprehensive water vapor condensation and collection scheme, greatly reducing the water loss typical to algae growing processes.

12. Oxygen released by the algae is vented or collected at strategically spaced locations throughout the system.

13. Nutrients are added into the tanks at the ends of the tubes.

14. Carbon dioxide collected by the scrubber may be fed to the system as part of a salt complex in a constant stream of reagent solution.

15. Algae are circulated using low abrasion devices that include auger style pumps.

16. Algae and spent solution are continuously drawn from the tubes. Reagent solution depleted of CO2 by the algae. Depleted reagent solution is pumped to the scrubber to be recharged.

17. Algae are removed (preferably using horizontal vacuum belt filters). The algae are washed with fresh water while it is on the filter belt. (This is a common practice with belt filters.)

18. The filtrate may be pumped to the scrubber in as integrated system. In the case where sodium carbonate is the scrubbing solution, a flow with concentrated sodium carbonate is converted into sodium bicarbonate in the scrubber.

19. The PBR with roof panels of claim 2, were in the roof panels are insulated.

20. A PBR cleaning system using balls slightly smaller than the inside diameter of the tubes. These balls will be slightly buoyant and will continuously be circulated in the PBR system removing the algae that might otherwise stick to the inside of these tubes. Tests have shown that these slightly buoyant balls will enter all tubes in the system randomly so that all tubes remain clean, an important feature, that assures all algae have exposure to optimum sunlight
